(19) United States
(12) Patent Application Publication
(10) Pub. No.: US 2008/0033311 A1
(43) Pub. Date: Feb. 7, 2008

(54) System And Method For Relating An Individual's Heart Rate And Power Output During Exercise

(76) Inventor: Jeffrey S. Sledge, Madison, WI (US)

Correspondence Address: BOYLE FREDRICKSON S.C., 840 North Plankinton Avenue, MILWAUKEE, WI, 53203, US

(21) Appl. No.: 11/462,887

(22) Filed: Aug. 7, 2006

(51) Int. Cl. A61B 5/04

(52) U.S. Cl. 600/509

(57) ABSTRACT

A system and method for relating heart rate and power output during exercise includes subjecting an individual to a physical activity that involves maintaining power output at a substantially constant level during a time period while monitoring heart rate. For an initial portion of the time period, there is a generally parallel correlation between power output and heart rate. Subsequently, a separation point occurs at which heart rate increases at a relatively rapid rate while power output remains relatively constant, which provides an accurate indication of the individual's anaerobic threshold. The separation point may be used to determine an exercise regimen for the individual, which may be used in a variety of ways, including in weight loss or maintenance, athletic training, physical therapy, etc. The physical activity may involve operation of an item of exercise equipment, such as a cycling exerciser, which includes means for sensing power output by the individual.

### BACKGROUND AND SUMMARY OF THE INVENTION

This invention relates to a system and method for ascertaining a physical characteristic of an individual, and more particularly to such a system and method for correlating an individual's power output with the individual's heart rate to identify a characteristic, such as lactate threshold, that is indicative of a person's physical fitness or that can be used in a medical application.

Various fitness testing systems and methods are known for ascertaining the fitness level of an individual, which include determinations of an anaerobic threshold, maximum VO2 and maximum heart rate. Using these parameters, it is possible to develop a training or exercise schedule to increase the individual's fitness and performance capability.

Testing for such physical fitness parameters is difficult. For example, an anaerobic threshold test may involve the exertion of physical exercise over time, and periodic blood sampling and testing during the exercise session to measure the amount of lactate in the individual's blood. Another example is a Conconi test, which involves plotting an individual's running speed against heart rate for predetermined distance intervals. Other tests involve running or cycling at a level of maximum intensity for a predetermined time period, and regularly recording heart rate during the time period.

The above-noted anaerobic threshold testing protocols are either overly invasive, and therefore impractical, for a majority of individuals, or are relatively inaccurate. Certain known tests can only provide meaningful results if the individual is capable of performing an aerobic activity for a period of time, which is not always the case. Accordingly, there is a need for a fitness testing system and method that can be used by a wide variety of individuals of any fitness level, and that provides a relatively accurate determination of an individual's anaerobic or lactate threshold and the relationship of the individual's anaerobic or lactate threshold to power output over time.

It is an object of the present invention to provide a system and method for testing an individual's fitness in a non-invasive manner, while providing an accurate determination of the individual's fitness level using power output information over time. It is a further object of the present invention to provide such a system and method that does not require any previous conditioning on the part of the individual. Yet another object of the present invention is to provide such a system and method which enables the individual to determine his or her fitness level relatively easily. A still further object of the present invention is to provide such a system and method which can be carried out by using equipment that is relatively readily available.

In accordance with one aspect, the present invention contemplates a method of testing the fitness of an individual, which includes subjecting the individual to a physical activity that involves an output of power by the individual over a time period that includes an initial portion and a terminal portion, and maintaining the power output of the individual at a substantially constant level during at least the terminal portion of the time period. The method further involves monitoring the heart rate of the individual during the time period. The duration of the terminal portion of the time period is determined by the time at which the rate of increase of the heart rate of the individual exceeds a predetermined acceptable rate of increase, at which time the physical activity of the individual is stopped. During most of the time period, there is a generally parallel correlation between the power output and the heart rate of the individual. At a point during the terminal portion of the time period, a separation point occurs at which a non-parallel relationship is exhibited between the power output and heart rate of the individual. At the separation point, the individual's heart rate increases at a relatively rapid rate while the individual's power output remains relatively constant or becomes erratic, which provides an accurate indication of the anaerobic threshold of the individual. The separation point may be used to determine an exercise regimen for the individual, which may be used in a variety of ways, including in a weight loss or maintenance program, an athletic training program, a physical therapy program, etc.

The act of subjecting the individual to a physical activity that involves the output of power may be carried out by operation of an item of exercise equipment by the individual. In one form, the physical activity may be in the form of operation of a cycling device by the individual, in which the cycling device includes means for sensing power output by the individual. The cycling device may be a stationary exercise cycle, or any other exercise device that is capable of accurately and consistently sensing power applied by an individual in order to operate the exercise device.

In accordance with another aspect, the present invention contemplates a fitness test that includes a physical exertion component involving the output of power by an individual over a time period that includes an initial portion and a terminal portion, and monitoring the individual's power output during the time period. The fitness test also includes a heart rate monitoring component involving simultaneously monitoring the individual's heart rate during the time period, and a correlation component that relates the individual's heart rate to the power output of the individual over the time period. As noted previously, the duration of the terminal portion of the time period is determined by the time at which the heart rate of the individual reaches a predetermined maximum threshold. The correlation component of the test involves an initial generally parallel correlation between the power output and the heart rate of the individual during the initial portion of the time period, and a subsequent separation correlation at which a non-parallel relationship is exhibited between the power output and heart rate of the individual during the terminal portion of the time period. Also as noted above, the physical exertion component may be operation of an item of exercise equipment by the individual, and the item of exercise equipment may be a cycling device that includes means for sensing power output by the individual.

The invention also contemplates a fitness testing system, which includes means for subjecting an individual to a physical activity that involves an output of power by the individual over a time period. The fitness testing system also includes means for monitoring the power output of the individual over the time period, and means for simultaneously monitoring the heart rate of the individual over the time period. In this manner, a correlation can be observed between the power output and the heart rate of the individual during the time period. The means for subjecting the individual to a physical activity may be an item of exercise equipment, and the means for monitoring the power output of the individual may be in the form of a power sensing arrangement associated with the item of exercise equipment. The item of exercise equipment may be a cycling device, such as a stationary exercise cycle, which includes means for sensing the power applied by the individual to a pedal arrangement associated with the cycling device.

Various other features, objects and advantages of the invention will be made apparent from the following description taken together with the drawings.

### BRIEF DESCRIPTION OF THE DRAWINGS

The drawings illustrate the best mode presently contemplated of carrying out the invention.

In the drawings:

FIG. 1 is a representation of an individual and an item of exercise equipment, in the form of a cycling exerciser, that can be used to carry out the fitness testing system and method in accordance with the present invention;

FIG. 2 is a flow chart representation of operation of the system of FIG. 1 in determining an individual's fitness level;

FIG. 3 is a representative graph showing correlation between an individual's power output and heart rate using the fitness testing system and method of the present invention; and

FIGS. 4a-4d are actual graphs showing correlation between an individual's power output and heart rate using the fitness testing system and method of the present invention.

### DETAILED DESCRIPTION OF THE INVENTION

Broadly, the present invention involves subjecting an individual to exercise over a period of time, and monitoring both the individual's power output and heart rate during the time period. The individual's heart rate can be monitored in any conventional manner such as, for example, a chest strap heart rate monitor, a wrist worn heart rate monitor or a palm-type heart rate monitor incorporated into an item of exercise equipment.

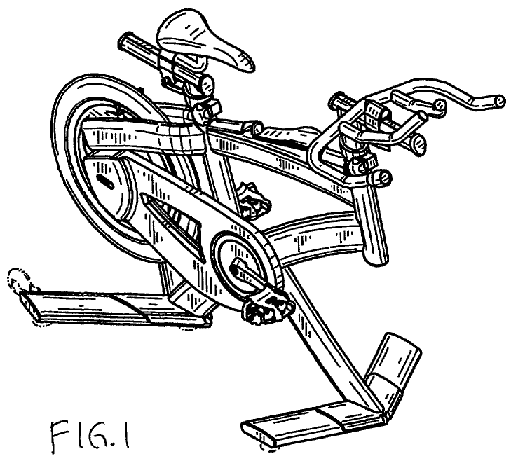

The power output monitoring component may be carried out using any type of device or exercise equipment capable of measuring the individual's power output during use of the device or equipment. Representatively, such devices or equipment may include a treadmill, a rowing exerciser, a swim stroke exerciser, a cycling exerciser, or a bicycle trainer, although it is understood that other devices or equipment may be employed. Preferably, the individual's power output is monitored in a controlled environment, using a stationary item of exercise equipment. The system and method of the present invention have been carried out using a stationary exercise cycle, such an exercise cycle manufactured and sold by Saris Cycling Group, Inc. of Madison, Wis. under its designation CYCLEOPS PRO 300PT Indoor Cycle, which includes a power sensing hub that measures torque applied by the individual in order to impart rotation to a flywheel. An exercise cycle of this type is illustrated in FIG. 1, and is shown and described in detail in copending patent application Ser. No. 11/192,506, filed Jul. 29, 2005, the disclosure of which is hereby incorporated by reference. The system and method of the present invention may also involve use of an adjustable resistance bicycle trainer, to which a bicycle fitted with a CYCLEOPS POWERTAP hub is mounted. In an arrangement of this type, a bicycle with a power sensing hub is used in conjunction with a variable resistance bicycle trainer.

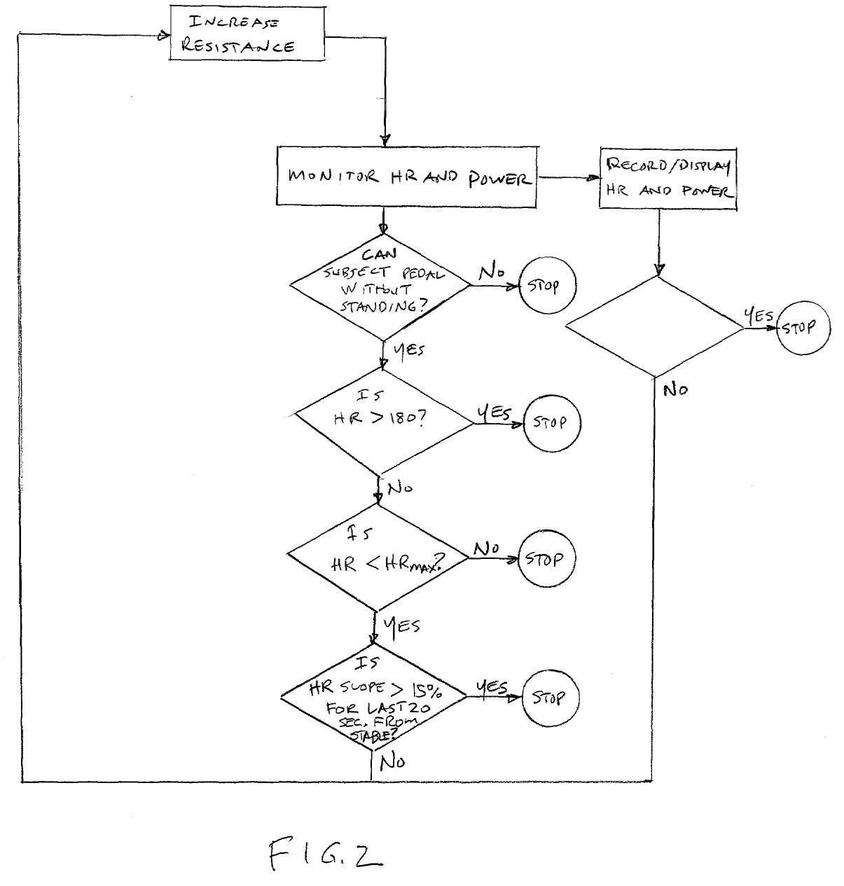

FIG. 2 generally illustrates the steps involved in the fitness testing system and method of the present invention. As shown in FIG. 2, the individual's heart rate and power output are continuously monitored. If desired, the individual's heart rate and power output may be recorded and/or displayed.

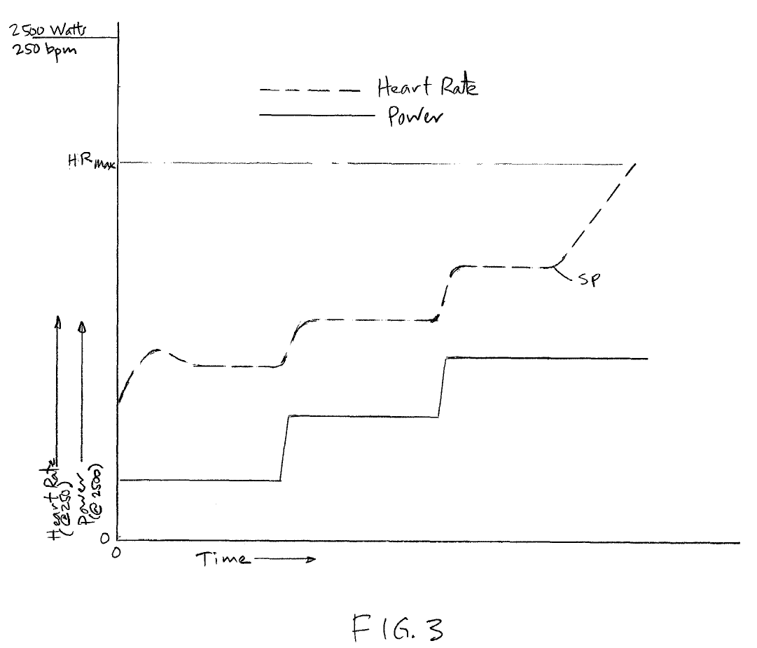

The system and method of the present invention involves a step-wise increase in the individual's power output for predetermined time periods, and monitoring the individual's heart rate throughout the time period. FIG. 3 is a representative graphic illustration of the relationship between the individual's heart rate and the power output of the individual using the system and method of the present invention.

During an initial warm-up phase, the individual's heart rate first peaks and then stabilizes at a relatively constant level while the individual applies a relatively low level of power. To accomplish the relatively constant output of power during the initial time period, the individual maintains a constant cadence (rate of pedal revolution). After the individual's heart rate has stabilized for a predetermined time period, the level of resistance applied to the exercise device is increased. The individual maintains the same cadence at the increased level of resistance as at the lower level of resistance, to provide an increased level of constant power output due to the increase in the level of resistance. Shortly following the increase in the resistance level, the individual's heart rate increases and then stabilizes. During both the initial warm-up phase and the phase of increased resistance, there is a relatively parallel relationship between the individual's heart rate and the individual's power output. After the individual's heart rate has again stabilized for a predetermined time period, the level of resistance applied by the exercise device is again increased, in approximately the same incremental degree of resistance increase as before. Again, the individual maintains a constant cadence at the increased resistance level, to provide an increase in power output so that a stable and constant power output is provided at the second increased level of resistance. The individual's heart rate again increases shortly after the resistance level is increased, and then stabilizes so as to again be generally parallel to the graph segment therebelow representing the individual's power output. If the individual's heart rate again stabilizes for the predetermined time period, the level of resistance is again increased while the individual maintains a constant cadence, to again raise the level of power output by the individual. Generally, the resistance increases are undertaken in equal increments in a step-wise fashion.

During a terminal phase of the time period, the individual's power output remains constant or becomes erratic, and the individual's heart rate departs from the parallel relationship relative to power output exhibited during the earlier phases. At this point (denoted as the separation point SP in FIG. 3), the individual's heart rate increases relatively rapidly until a maximum heart rate value is reached. When the rate at which the individual's heart rate reaches or exceeds the maximum rate of increase, the test is ended and the individual stops operation of the exercise device.

The separation point SP is believed to indicate the individual's anaerobic threshold, which provides valuable information as to the fitness level of the individual and can be used to determined exercise regimens for the individual in a variety of applications. Representatively, the exercise regimens may be in connection with a weight loss program, a weight maintenance program, an athletic training program, a physical therapy program, a cardiac rehabilitation program, an orthopedic rehabilitation program, etc. In a particularly useful application, this information can be used in an effort to combat obesity in overweight children, by combining prescriptive exercise regimens with bicycling, which is an activity commonly enjoyed by children. In any application, the present invention provides the ability to individualize a therapy or regimen to provide an individualized intervention for the benefit of a person. While the present invention has been shown and described with respect to use of a stationary cycle to measure power output, it is understood that the invention may also involve use of any controlled, measurable resistive activity in which conditions can be replicated. For example, any other stationary device such as a resistive treadmill, elliptical trainer, rowing exerciser, swim stroke exerciser, etc., may be employed, as long as it is possible to increase resistive force in a controlled manner and measure the user's power output. It is also considered that a non-stationary activity may be employed, e.g. riding a bicycle equipped with a resistance mechanism on a flat surface.

### EXAMPLE

In a representative example of the system and method of the present invention, adolescent children, ages 12-14, with a BMI>95 were selected from a pool of volunteers for an analysis and intervention, in connection with a longitudinal study of the health effects of a series of physical education regimes for children with, or tending to, obesity.

The study group participated in a four-compartment fitness assessment as well as the experimental power protocol of the present invention. The four-compartment fitness assessment test included: a blood draw, sub-maximal treadmill test, Dexa body scan, and Maximal VO2 treadmill test.

The study made use of the CycleOps Pro 300PT Indoor Cycle (IC) manufactured by Saris Cycling Group of Madison, Wis. The drive train of the IC includes a PowerTap sensor in a fixed gear assembly. The PowerTap sensor is contained within a 45 lb. free wheel to which the rear cog is bolted. The rear cog is directly coupled to the chain wheel and cranks of the IC. No freewheel assembly is used. Independent testing has established PowerTap measurement devices to be 98.5% accurate.

The IC measures exerted instantaneous force at the rear hub. This force is reported in watts of angular torque. The power measurement device is made up of four solid-state strain gauges attached to a torque tube. As force is applied to the torque tube by pedaling, the tube twists and the resistance of the strain gauges varies. A CPU within the hub converts the change in electrical resistance into discrete values and records those values over time. The time values of force (work) are recorded and displayed in Kilojoules of energy expended. A digital radio frequency transmitter sends the instantaneous torque and time values along with flywheel rotational speed to a handle bar mounted CPU.

Test subjects wore a heart rate monitor strap that transmitted heart rate to the CPU on a separate radio channel. Heart rate was monitored both on the CPU display and on a heart rate monitor watch worn by the test administrator. Having the heart rate available on a discrete heart rate watch served a non-invasive safety function, in that subjects could be monitored without taking heart rate directly and without interfering with test performance.

The handle bar mounted CPU included a microprocessor, memory, display and display controls, as well as ports for transporting data to an external personal computer. The CPU can be configured for different recording rates and is capable of storing several hours of data. For this test, all data elements were recorded at a one second interval. The data display reads out the data elements as they come into the CPU. The display is visible to the test subject, whose cooperation in reading and working with the display was necessary for this test.

Data recorded during the power protocol test was downloaded to a personal computer for post processing and visualization. The CPU was cleared, re-set and tested after each subject was tested.

At the conclusion of the four-compartment test, each subject was given 10-15 minutes of active cool down. The target elapsed time from the end of the maximal VO2 test to the beginning of the power protocol was twenty minutes. An IC compatible heart rate monitor strap was attached to each subject and its function verified. Each subject was introduced to the IC and the basic nature of the test.

The IC was adjusted to fit each subject. Proper saddle position, knee/pedal spindle position, handlebar height and reach were established. After initial positioning, the subject was asked to spin slowly so that body mechanics could be checked. If needed, final positioning adjustments were made. Subjects were asked to continue spinning slowly while the CPU display was explained. Each subject was shown his or her power output, heart rate and cadence on the CPU display. In the event of a subject being tested more than once, the IC must be adjusted to ensure that the subject has the same body mechanics and position as in all prior tests, to ensure consistency in test results.

The protocol is cadence based. Subjects were asked to pedal at a consistent 55 rpm throughout the test. Each subject would observe his or her cadence rate and make adjustments to speed up or slow down as needed to hold a predetermined cadence. All subjects (with the exception of one with known cognitive difficulties) were able to hold cadence at an acceptable level (within a range of +/−5 rpm). Prior research indicated that this group had difficulty pedaling at higher cadences. Lack of experience, coordination and large leg sizes made high efficiency cadence rates of 85 to 105 rpm difficult to achieve and maintain. Cadence rates below 50 rpm have the potential of creating high joint stress at high wattage.

With adjustments made and the cadence/display relationship understood, subjects were asked to spin in the cadence range for a warm-up period. No load was placed on the IC flywheel and the effort required to spin the flywheel at 55 rpm was in the 25 to 30 watt range. Subjects were asked to spin for five minutes or until heart rate stabilized for thirty seconds.

A resistive load was then placed on the flywheel that required an additional 20 watts of energy to keep it spinning at the proper cadence. A one-minute time period for adjustment and heart rate stabilization was allowed at this new load. The test continued with the addition of 20 watts every two minutes (plus one minute for adjustment) until one of three conditions were met:

Subject's heart rate exceeded 180 bpm (a predetermined safety limit);

Heart rate “separated” from produced power and exhibited a rising slope; or

Subject was unable to turn the cranks because of high loads.

Throughout the test, subjects were shown a Borg Reported Perceived Exertion (0-20) scale and asked about perceived exertion. Responding to the Borg scale did not interfere with their ability to maintain cadence.

All tests were completed within twenty minutes, with the majority of tests completed in fifteen minutes. At the point at which one of the maximal conditions for the test was met, all subjects reported an RPE of 16-17 regardless of which condition was met. During the test there was a distinct male/female differentiation in response. Female subjects respond to the Borg scale in-line with demonstrated heart rates and power output. Male subjects consistently under-reported RPE until the point at which one of the maximal test conditions was reached.

Each subject's heart rate exhibited a parallel relationship with power output in a stepwise fashion as the resistive load increased, until the subject approached what appears to be the subject's anaerobic threshold. At threshold, heart rate “separated” from power (power being a horizontal value) and began to show a rise in slope. The study group demonstrated wide separation between heart rate and power production. Plotted power production was substantially below heart rate. This is in contrast to fit individuals where plotted power output is close to or exceeds heart rate From an analysis of the data, it is possible to develop individual ratios of heart rate to power that track the efficiency of the body's transport mechanism in relationship to its ability to produce motive power. When combined with subject weight, this ratio can be used to inform subject “condition.”

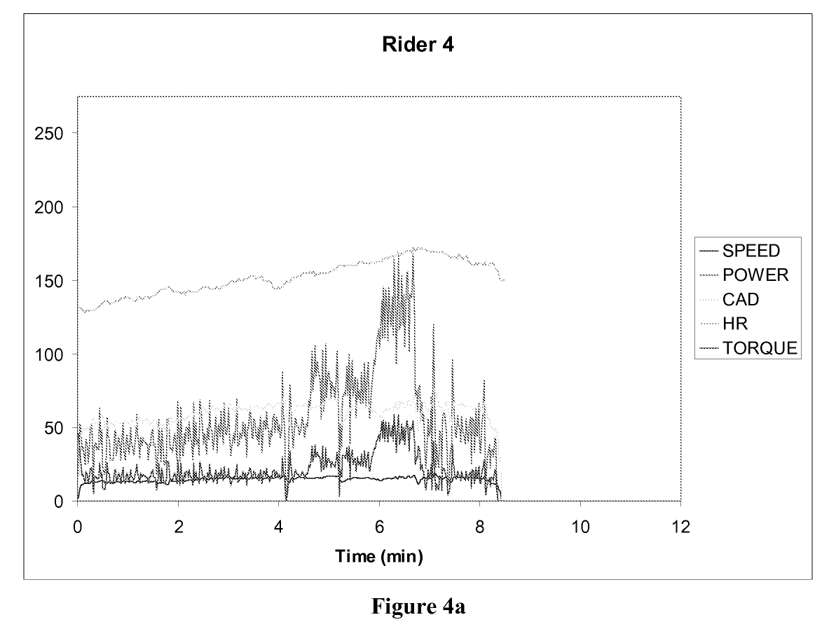

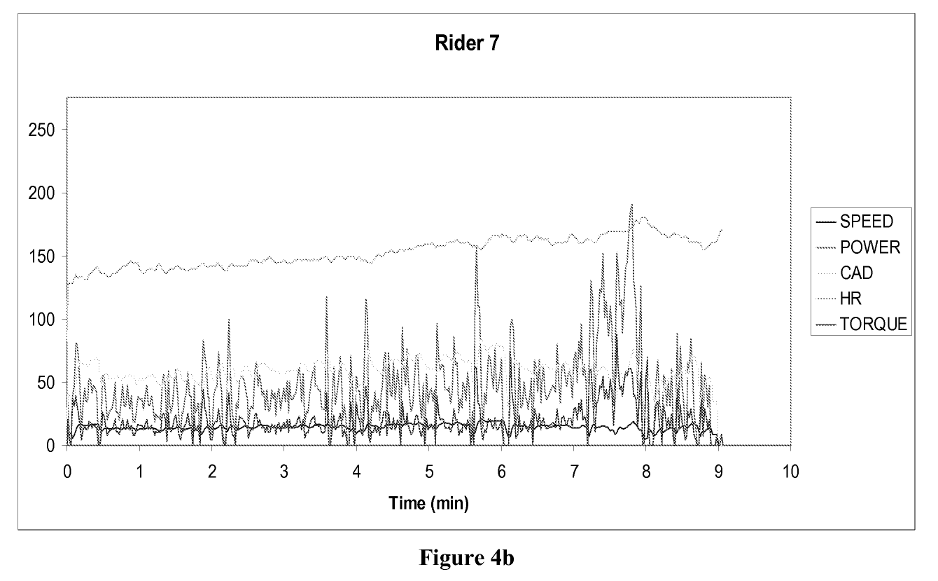

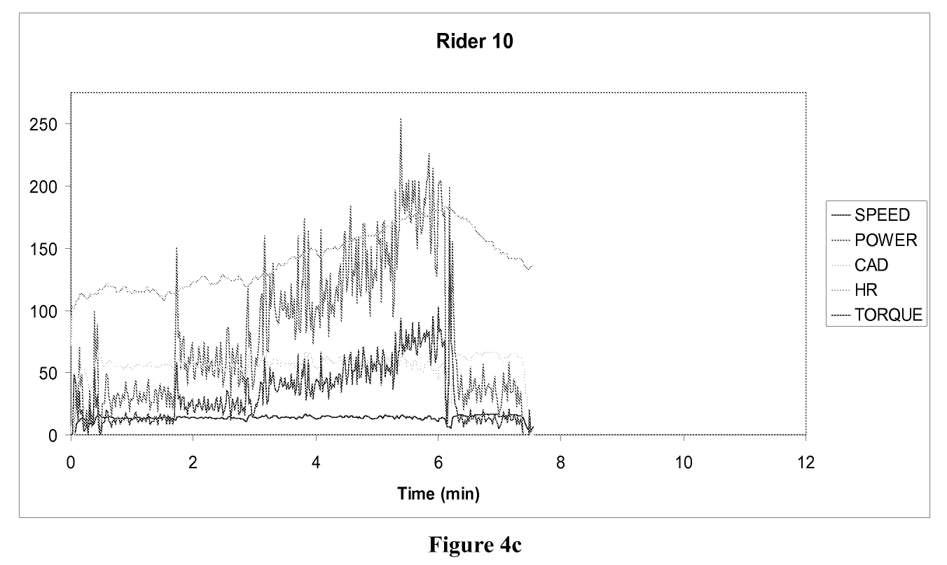

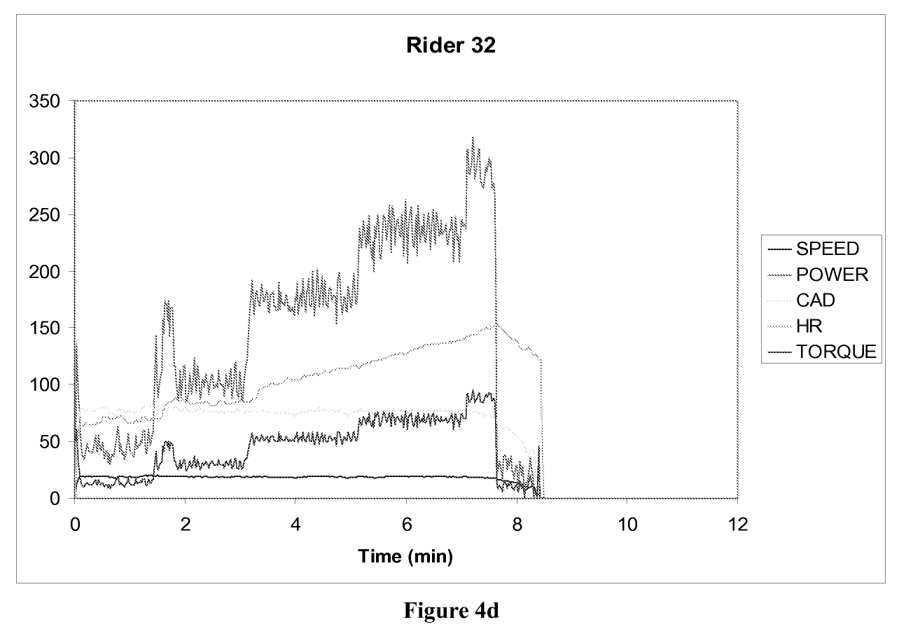

FIGS. 4a through 4d are graphs that show test results for several actual test subjects, and which illustrate the correlation noted previously. The graph of FIG. 4a shows the results of testing a person having a generally low fitness level, in which power output is somewhat erratic but in which the noted correlation between power output and heart rate is present. The graph of FIG. 4b shows the results of testing a person having an exceptionally poor fitness level, again showing an erratic power output but the noted correlation between power output and heart rate. The graph of FIG. 4c shows the results of testing a person having a marginal fitness level, again showing the noted correlation between power output and heart rate. The graph of FIG. 4d shows the results of testing an exceptionally fit person, in which the noted correlation between power output and heart rate is clear.

If used to develop standard “zone” divisions for power production (percentage of lactate threshold) the aggregate data for the cohort indicates a maximal fat burning for this group should occur at 30-45 watts of continuous power production. This equates to cycling at roughly 4-7 mph on level ground in a moderate breeze. This level of power production is far lower than expected prior to this study, but in-line with the data from this test.

The test protocol of particular value in establishing the metrics necessary for individualized interventions for weight loss, building cardiovascular condition, and strength and flexibility training. The ratio of heart rate (stress) to power (strain) provides a relative measure of fitness of the individual when coupled with an understanding of the individual's sustained and peak power production capabilities. The power protocol is also adaptable to general populations by adjusting the starting and/or incremental increases in power. With small populations of adult fit cyclists the protocol seemed to parallel other findings and observations on their general condition.

With the ability to precisely measure the power production of adolescent bicyclist as they ride and play day to day, it is possible to design prescriptive routes to address individual needs.

Various alternatives and embodiments are contemplated as being within the scope of the following claims particularly pointing out and distinctly claiming the subject matter regarded as the invention.

I claim:

1. A method of testing the fitness of an individual, comprising the acts of:
subjecting the individual to a physical activity that involves an output of power by the individual over a time period that includes an initial portion and a terminal portion;
maintaining the power output of the individual at a substantially constant level during at least the terminal portion of the time period; and
monitoring the heart rate of the individual during the time period.

2. The method of claim 1, wherein the end of the terminal portion of the time period is determined by the time at which the rate of increase of the individual's heart rate reaches a predetermined threshold.

3. The method of claim 1, including the acts of observing a correlation between the power output and the heart rate of the individual during the initial portion of the time period, and observing a separation point during the terminal portion of the time period at which a non-parallel relationship is exhibited between the power output and heart rate of the individual.

4. The method of claim 3, further comprising the act of utilizing the separation point to determine an exercise regimen for the individual.

5. The method of claim 4, wherein the exercise regimen is associated with a program for the individual selected from the group consisting of a weight loss program, a weight maintenance program, an athletic training program, a physical therapy program, a medical testing program, and a medical rehabilitation program.

6. The method of claim 1, wherein the act of subjecting the individual to a physical activity that involves the output of power is carried out by operation of an item of exercise equipment by the individual.

7. The method of claim 6, wherein the act of subjecting the individual to a physical activity that involves the output of power is carried out by operation of a cycling device by the individual, wherein the cycling device includes means for sensing power output by the individual.

8. The method of claim 7, wherein the act of subjecting the individual to a physical activity that involves the output of power is carried out by operation of a stationary cycling device by the individual.

9. A fitness test, comprising:
a physical exertion component involving the output of power by an individual over a time period including an initial portion and a terminal portion, and monitoring the individual's power output during the time period;
a heart rate monitoring component involving simultaneously monitoring the individual's heart rate during the time period; and
a correlation component that relates the individual's heart rate to the power output of the individual over the time period.

10. The fitness test of claim 9, wherein the duration of the terminal portion of the time period is determined by the time at which the heart rate of the individual reaches a predetermined threshold.

11. The fitness test of claim 9, wherein the correlation component of the test involves an initial generally parallel correlation between the power output and the heart rate of the individual during the initial portion of the time period, and a subsequent separation correlation at which a non-parallel relationship is exhibited between the power output and heart rate of the individual during the terminal portion of the time period.

12. The fitness test of claim 11, wherein the duration of the time period is determined by the time at which the heart rate of the individual reaches a predetermined threshold during the separation correlation.

13. The fitness test of claim 9, wherein the physical exertion component comprises operation of an item of exercise equipment by the individual.

14. The fitness test of claim 13, wherein the item of exercise equipment comprises a cycling device that includes means for sensing power output by the individual.

15. The fitness test of claim 14, wherein cycling device comprises a stationary cycling device.

16. A fitness testing system, comprising:
means for subjecting an individual to a physical activity that involves an output of power by the individual over a time period;
means for monitoring the power output of the individual over the time period; and
means for simultaneously monitoring the heart rate of the individual over the time period.

17. The fitness testing system of claim 16, further including means for correlating the power output and the heart rate of the individual during the time period.

18. The fitness testing system of claim 17, wherein the means for subjecting the individual to a physical activity comprises an item of exercise equipment, and wherein the means for monitoring the power output of the individual is associated with the item of exercise equipment.

19. The fitness testing system of claim 17, wherein the item of exercise equipment comprises a cycling device and wherein the means for monitoring the power output of the individual comprises means for sensing the power applied by the individual to a pedal arrangement associated with the cycling device.

20. The fitness testing system of claim 19, wherein the cycling device comprises a stationary exercise cycle.
